(19) United States
(12) Patent Application Publication
(10) Pub. No.: US 2026/0124183 A1
(43) Pub. Date: May 7, 2026

(54) METHODS OF TREATING SPINOCEREBELLAR ATAXIAS

(71) Applicant: Biohaven Therapeutics Ltd., New Haven, CT (US)

(72) Inventors: Vladimir CORIC, Madison, CT (US); Melissa BEINER, Madison, CT (US); Irfan QURESHI, Hackensack, NJ (US); Gilbert L'ITALIEN, Deep River, CT (US)

(21) Appl. No.: 18/867,416

(22) PCT Filed: May 23, 2023
(86) PCT No.: PCT/US2023/067326

§ 371 Date: Nov. 19, 2024

Related U.S. Application Data
(60) Provisional application No. 63/344,917, filed on May 23, 2022.

(51) Int. Cl. A61K 31/428; A61K 9/00; A61P 25/28

(52) U.S. Cl. CPC A61K 31/428; A61K 9/0053; A61P 25/28

(57) ABSTRACT

Provided is a method for treating spinocerebellar ataxia genotype type 3 in a patient in need thereof, including administering to the patient a dosage form comprising an effective amount of troriluzole hydrochloride monohydrate. Also provided is a dosage form including troriluzole hydrochloride monohydrate in an amount effective to treat spinocerebellar ataxia genotype type 3 in a patient in need thereof.

### CROSS-REFERENCE TO RELATED APPLICATIONS

This application claims priority to U.S. Provisional Application No. 63/344,917 filed May 23, 2023, and all the benefits accruing therefrom under 35 U.S.C. § 119, the content of which is herein incorporated in its entirety by reference.

### FIELD OF THE INVENTION

The present invention relates to methods of treating spinocerebellar ataxias. In particular, this application relates to methods of treating spinocerebellar ataxia genotype type 3.

### BACKGROUND OF THE INVENTION

Ataxia is a disorder of the central nervous system, wherein the patient is unable to coordinate muscles for the execution of voluntary movement, see, e.g., Klockgether, T. “Ataxias” in Parkinsonism and Related Disorders 13, S391-S394, 2007. Typical symptoms of ataxia are gait dysfunctions, imbalance, impaired limb coordination and altered speech. In many ataxia disorders, the ataxia is due to degeneration of the cerebellar cortex and its afferent or efferent fiber connections. Typical affected brain regions are cerebellum, posterior column, pyramidal tracts and basal ganglia. Ataxia may lead to a decreased motoneuron function.

Spinocerebellar ataxia (“SCA”) is a group of inherited brain disorders, which affects cerebellum, a part of brain vital to coordination of physical movement, and sometimes spinal cord. Spinocerebellar ataxia can be classified into ataxias associated with translated GAG repeat expansions (SCA types 1, 2, 3, 6, 7 and 17), ataxias associated with untranslated repeat expansions in non-coding regions (SCA types 10 and 12), ataxias associated with point-mutations (SCA types 5, 13, 14 and 27).

The most common SCAs include type 1, 2, 3, 6, 7, 8 and 10. SCA1 often produces gait ataxia, limb ataxia, and dysarthria, with brainstem involvement but little cognitive abnormality. SCA2 is notable for the association of ataxia and dysarthria with slow saccadic eye movements and polyneuropathy. SCA3 (also known as Machado-Joseph disease) is often accompanied by eyelid retraction, reduced blinking, external ophthalmoplegia, dysarthria, dysphagia, and sometimes parkinsonism or peripheral neuropathy. SCA6 is comparatively less severe, typically progresses more slowly, is more limited to cerebellar involvement than other SCAs, and has a later age of onset. SCA7 is distinguished by retinal degeneration leading to blindness, in addition to ataxia. Overall, there is significant symptom overlap among these SCAs. The shared symptomatic manifestations of the SCAs may reflect common pathology affecting cerebellar purkinje cell fibers.

There are currently no United States Food and Drug Administration (“FDA”) approved medications for the treatment of SCAs. There remains a need for effective medications to treat various types of SCA as well as a broader spinocerebellar ataxia population.

### SUMMARY OF THE INVENTION

The present invention is directed to methods of treating spinocerebellar ataxia genotype type 3 and dosage forms for such treatment.

In an embodiment, provided is a method for treating spinocerebellar ataxia genotype type 3 in a patient in need thereof, including administering to the patient a dosage form comprising an effective amount of troriluzole hydrochloride monohydrate.

In another embodiment, provided is a dosage form including troriluzole hydrochloride monohydrate in an amount effective to treat spinocerebellar ataxia genotype type 3 in a patient in need thereof.

### BRIEF DESCRIPTION OF THE DRAWINGS

These and/or other aspects will become apparent and more readily appreciated from the following description of the embodiments, taken in conjunction with the accompanying drawings in which:

FIG. 1 a graph showing f-SARA change from randomization baseline to week 48 in f-SARA total score by treatment arm among all SCA patients (nominal p=0.76);

FIG. 2 is a graph showing f-SARA change from randomization baseline to week 48 in f-SARA total score by treatment arm among SCA3 patients (with baseline f-SARA Gait Item score included as covariate, nominal p=0.53);

FIG. 3 is a graph showing f-SARA change from randomization baseline to week 48 in f-SARA total score by treatment arm among SCA3 patients who could walk without assistance at baseline (nominal p=0.031); and

FIG. 4 is a table showing frequency of treatment-emerging adverse events (TEAE) of fall among various patient groups.

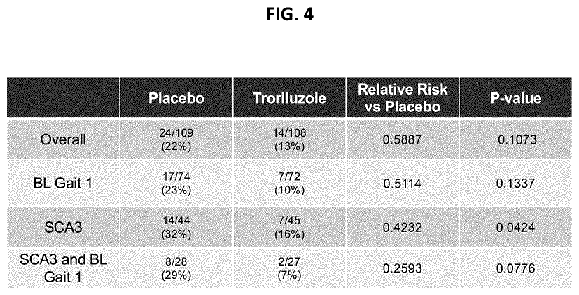

### DETAILED DESCRIPTION OF THE INVENTION

The following detailed description is provided to aid those skilled in the art in practicing the present invention. Exemplary embodiments will hereinafter be described in detail. However, these embodiments are only exemplary, and the present disclosure is not limited thereto but rather is defined by the scope of the appended claims. Those of ordinary skill in the art may make modifications and variations in the embodiments described herein without departing from the spirit or scope of the present disclosure.

Accordingly, the embodiments are merely described below, by referring to structures and schemes, to explain aspects of the present description. As used herein, the term “and/or” includes any and all combinations of one or more of the associated listed items. The term “or” means “and/or.” Expressions such as “at least one of,” when preceding a list of elements, modify the entire list of elements and do not modify the individual elements of the list.

It will be understood that when an element is referred to as being “on” another element, it can be directly in contact with the other element or intervening elements may be present therebetween. In contrast, when an element is referred to as being “directly on” another element, there are no intervening elements present.

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the present embodiments.

It is understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all technical and scientific terms used herein have the same meaning as commonly understood by one of ordinary skill in the art to which this disclosure belongs. The terminology used in the description is for describing particular embodiments only and is not intended to be limiting. It will be further understood that the terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used in this application, except as otherwise expressly provided herein, each of the following terms shall have the meaning set forth below. Additional definitions are set forth throughout the application. In instances where a term is not specifically defined herein, that term is given an art-recognized meaning by those of ordinary skill applying that term in context to its use in describing the present invention.

The articles “a” and “an” refer to one or to more than one (i.e., to at least one) of the grammatical object of the article unless the context clearly indicates otherwise. By way of example, “an element” means one element or more than one element.

Additional aspects will be set forth in part in the description which follows and, in part, will be apparent from the description.

The starting materials useful for making the pharmaceutical compositions of the present invention are readily commercially available or can be prepared by those skilled in the art.

In an embodiment, provided is a method for treating spinocerebellar ataxia genotype type 3 in a patient in need thereof, including administering to the patient a dosage form comprising an effective amount of troriluzole hydrochloride monohydrate.

In an aspect, the dosage form may be in the form of a capsule. In another aspect, the dosage form may be in the form of a tablet. The tablet may be an orally disintegrating tablet.

Troriluzole hydrochloride monohydrate is a member of the benzothiazole chemical class and is a tripeptide prodrug conjugate of riluzole. Troriluzole hydrochloride monohydrate is described chemically as 2-amino-N-({methyl-[(6-trifluoromethoxy-benzothiazol-2-ylcarbamoyl)-methyl]-carbamoyl}-methyl)-acetamide monohydrate monohydrochloride and its structural formula is:

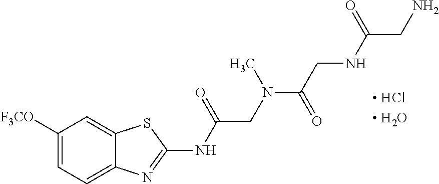 [SMILES: CN(CC(=O)Nc1nc2ccc(OC(F)(F)F)cc2s1)C(=O)CNC(=O)CN]

The molecular formula of troriluzole hydrochloride monohydrate is C₁₅H₁₆F₃N₅O₄S·HCl·H₂O, representing a molecular weight of 473.85 g/mol (anhydrous free base form molecular weight is 419.40 g/mol). The drug substance is freely soluble in water.

In an aspect, the dosage form may include 200 mg of troriluzole hydrochloride monohydrate, and may be administered to the patient daily (QD). In another aspect, the dosage form may include 100 mg of troriluzole hydrochloride monohydrate, and may be administered to the patient twice a day (BID).

In an aspect, the dosage form may be administered daily for at least forty-eight weeks. For example, the dosage form may be administered daily for forty-eight weeks. When the dosage form including 200 mg of troriluzole hydrochloride monohydrate is administered to the patient daily for forty eight weeks, the patient at week 48 may show a least squares [LS] mean change difference of −0.55 compared to placebo (nominal p-value=0.053, 95% Cl: −1.12, 0.01). When the dosage form including 200 mg of troriluzole hydrochloride monohydrate is administered to the patient, who was able to walk without assistance at baseline, daily for forty eight weeks, the patient at week 48 may show a least squares [LS] mean change difference of −0.71 compared to placebo (nominal p-value=0.031, 95% Cl: −1.36, −0.07).

In another aspect, the dosage form may be administered daily for at least four weeks. For example, the dosage form may be administered daily for four weeks. When the dosage form including 200 mg of troriluzole hydrochloride monohydrate is administered to the patient daily for four weeks, the patient at week 4 may show a least squares [LS] mean change difference of at least −0.46 compared to placebo. When the dosage form including 200 mg of troriluzole hydrochloride monohydrate is administered to the patient, who was able to walk without assistance at baseline, daily for four weeks, the patient at week 4 may show a least squares [LS] mean change difference of at least −0.67 compared to placebo.

In another aspect, the dosage form may be administered daily for at least eight weeks. For example, the dosage form may be administered daily for eight weeks. When the dosage form including 200 mg of troriluzole hydrochloride monohydrate is administered to the patient daily for eight weeks, the patient at week 8 may show a least squares [LS] mean change difference of at least −0.12 compared to placebo. When the dosage form including 200 mg of troriluzole hydrochloride monohydrate is administered to the patient daily for eight weeks, the patient at week 8 may show a least squares [LS] mean change difference of at least −0.33 compared to placebo.

In another aspect, the dosage form may be administered daily for at least twelve weeks. For example, the dosage form may be administered daily for twelve weeks. When the dosage form including 200 mg of troriluzole hydrochloride monohydrate is administered to the patient daily for twelve weeks, the patient at week 12 may show a least squares [LS] mean change difference of at least −0.46 compared to placebo. When the dosage form including 200 mg of troriluzole hydrochloride monohydrate is administered to the patient daily for twelve weeks, the patient at week 12 may show a least squares [LS] mean change difference of at least −0.71 compared to placebo.

In another aspect, the dosage form may be administered daily for at least twenty-four weeks.

For example, the dosage form may be administered daily for twenty-four weeks. When the dosage form including 200 mg of troriluzole hydrochloride monohydrate is administered to the patient daily for twenty-four weeks, the patient at week 24 may show a least squares [LS] mean change difference of at least −0.17 compared to placebo. When the dosage form including 200 mg of troriluzole hydrochloride monohydrate is administered to the patient daily for twenty-four weeks, the patient at week 24 may show a least squares [LS] mean change difference of at least −0.40 compared to placebo.

In another aspect, the dosage form may be administered daily for at least thirty-six weeks. For example, the dosage form may be administered daily for thirty-six weeks. When the dosage form including 200 mg of troriluzole hydrochloride monohydrate is administered to the patient daily for thirty-six weeks, the patient at week 36 may show a least squares [LS] mean change difference of at least −0.72 compared to placebo. When the dosage form including 200 mg of troriluzole hydrochloride monohydrate is administered to the patient daily for thirty-six weeks, the patient at week 36 may show a least squares [LS] mean change difference of at least −0.55 compared to placebo.

In another aspect, the dosage form may be administered daily for forty-eight weeks, and then administered daily again for an additional forty-eight weeks.

In another embodiment, provided is a dosage form including troriluzole hydrochloride monohydrate in an amount effective to treat spinocerebellar ataxia genotype type 3 in a patient in need thereof. In an aspect, the dosage form may include troriluzole hydrochloride monohydrate in an amount of 100 mg or greater. In another aspect, the dosage form may include troriluzole hydrochloride monohydrate in an amount of 140 mg or greater. In yet another aspect, the dosage form may include troriluzole hydrochloride monohydrate in an amount of 200 mg or greater.

The invention is further illustrated by the following non-limiting examples.

### Examples

Phase 3 Clinical Study for Troriluzole in Adult Subjects with Spinocerebellar Ataxia

### Brief Summary:

The purpose of this study is to compare the efficacy of Troriluzole (200 mg once daily) versus placebo after 48 weeks of treatment in subjects with spinocerebellar ataxia (SCA).

### Condition or Disease:

Spinocerebellar Ataxias
Spinocerebellar Ataxia Type 1
Spinocerebellar Ataxia Type 2
Spinocerebellar Ataxia Type 3
Spinocerebellar Ataxia Type 6
Spinocerebellar Ataxia Type 7
Spinocerebellar Ataxia Type 8
Spinocerebellar Ataxia Type 10

### Intervention/Treatment:

Troriluzole
Placebo

### Study Design:

Study Type: Interventional (Clinical Trial)
Actual Enrollment: 218 participants
Allocation: Randomized
Intervention Model: Parallel Assignment
Masking: Triple (Participant, Care Provider, Investigator)
Primary Purpose: Treatment
Official Title: A Phase III, Long-Term, Randomized, Double-blind, Placebo-controlled Trial of Troriluzole in Adult Subjects With Spinocerebellar Ataxia.

### Arms and Interventions:

Arm
Experimental: Arm 1: BHV-4157
Troriluzole 200 mg by mouth
Placebo Comparator: Arm 2: Placebo
Placebo 200 mg by mouth
Intervention/Treatment
Drug: troriluzole
200 mg by mouth
Drug: placebo
200 mg by mouth

### Outcome Measures

### Primary Outcome Measures

1. Change from Baseline in the total score of the Modified Functional Scale for the Assessment and Rating of Ataxia (f-SARA) after 48 weeks of treatment. [Time Frame: Baseline to week 48]
   An increase in the total score indicates a worsening of symptoms.

### Secondary Outcome Measures

1. Change from baseline in Patient Impression of Function and Activities of Daily Living Scale (PIFAS) score at Randomization Phase Week 48. [Time Frame: Baseline to week 48]
   An increase in the total score indicates a worsening of symptoms.
2. Change from baseline in Activities of Daily Living Scale from the Friedreich's Ataxia Rating Scale (FARS-ADL) at Randomization Week 48. [Time Frame: Baseline to week 48]
   An increase in the total score indicates a worsening of symptoms.
3. Change from baseline in Functional Staging for Ataxia from the Friedreich's Ataxia Rating Scale (FARS-FUNC) at Randomization Phase Week 48. [Time Frame: Baseline to week 48]
   An increase in the total score indicates a worsening of symptoms.
4. Frequency of subjects with the following adverse events (AEs) identified from case report forms: AEs (by severity; by relationship to study drug; overall); SAEs; and AEs leading to treatment discontinuation. [Time Frame: Baseline to week 48]

### Eligibility Criteria

### Inclusion Criteria

1. Subjects with a known or suspected diagnosis of the following specific hereditary ataxias: SCA1, SCA2, SCA3, SCA6, SCA7, SCA8 and SCA10; currently only enrolling SCA 1, SCA2, SCA3, SCA7, and SCA10 (the cap has been met for SCA6 and SCA8 (on May 31, 2019));
   a. A subject should have a confirmed genotypic diagnosis from a CLIA certified lab (can produce test results); or
   b. A subject has a family member that has a confirmed genotypic diagnosis from a CLIA certified lab (can produce test results) and must be willing to undergo genetic testing to confirm underlying SCA diagnosis; or
   c. A subject has a confirmed genotypic diagnosis from a lab that is not CLIA certified and must be willing to undergo genetic testing to confirm underlying SCA diagnosis; or
   d. A subject has clinical evidence that supports diagnosis of one of the aforementioned SCA genotypes but does not have producible test results from a CLIA certified lab from either a family member or for his or herself and the subject must be willing to undergo such testing to confirm the SCA diagnosis (in this case, site must wait for results of genotypic testing prior to randomization).
2. Ability to ambulate 8 meters without human assistance (canes and other devices allowed).
3. Screening f-SARA total score ≥3.
4. Score of ≥1 on gait subsection of the f-SARA.
5. Determined by the investigator to be medically stable at Baseline/randomization as assessed by medical history, physical examination, laboratory test results, and electrocardiogram testing.

### Exclusion Criteria

1. A ≥2-point difference on the Modified Functional SARA score between screening and baseline.
2. MMSE score <24.
3. Any medical condition other than one of the hereditary ataxias specified in the inclusion criteria that could predominantly explain or contribute significantly to the subjects' symptoms of ataxia.
4. A prominent spasticity or dystonia that, in the opinion of the investigator, will compromise the ability of the SARA instrument to assess underlying ataxia severity.
5. A score of 4 on any individual item (Items 1-4) of the f-SARA.
6. Subjects should be excluded at screening or baseline if medical conditions have arisen or there is a change in disease status that could confound the ability of the SARA to accurately reflect changes in ataxia severity.
7. Active liver disease or a history of hepatic intolerance to medications that in the investigator's judgment, is medically significant.

### Results

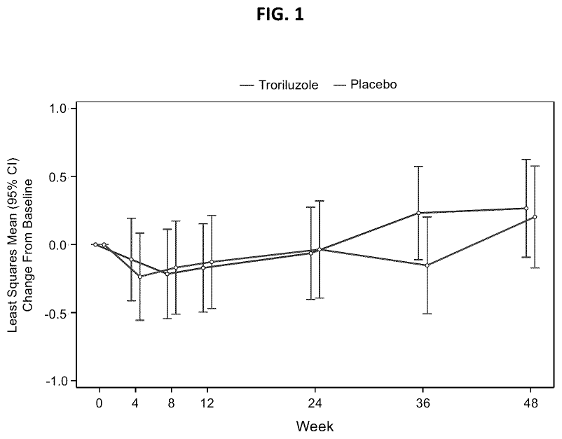

The primary endpoint, change from baseline to Week 48 on the modified functional Scale for the Assessment and Rating of Ataxia (f-SARA), did not reach statistical significance in the overall SCA population as there was less than expected decline is disease progression over the course of the study. In the overall study population (N=213), the troriluzole and placebo groups each had mean baseline scores of 4.9 on the f-SARA and the two groups showed minimal change at the 48-week endpoint with f-SARA scores of 5.1 and 5.2, respectively (p=0.76). The results are shown in FIG. 1.

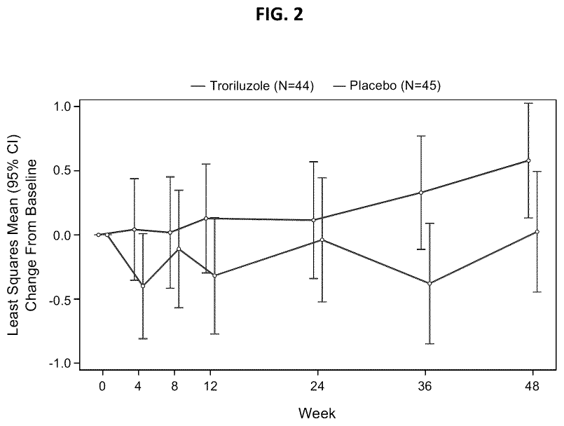

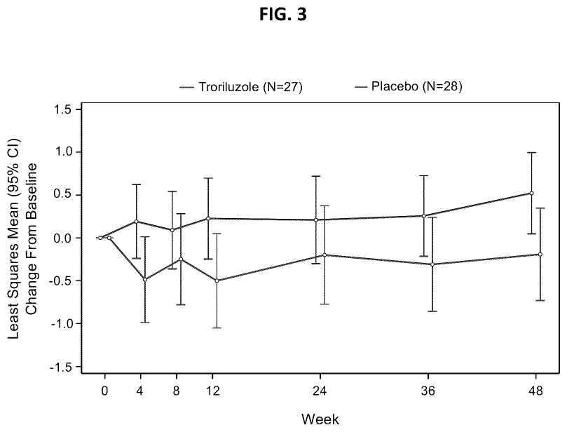

Post-hoc analysis of efficacy measures by genotype suggest a treatment effect in patients with the SCA Type 3 (SCA3) genotype, which represents the most common form of SCA. In the SCA3 subgroup (FIG. 2), troriluzole showed a numerical treatment benefit on the change in f-SARA score from baseline to Week 48 compared to placebo (least squares [LS] mean change difference −0.55, nominal p-value=0.053, 95% Cl: −1.12, 0.01). Further, in patients in the SCA3 subgroup who were able to walk without assistance at baseline (i.e., f-SARA Gait Item score=1) (FIG. 3), troriluzole demonstrated a greater numerical treatment benefit on the change in f-SARA score from baseline to Week 48 compared to placebo (LS mean change difference −0.71, nominal p-value=0.031, 95% Cl: −1.36, −0.07). Notably, the f-SARA is a novel, 16-point scale developed in collaboration with FDA as the primary outcome measure for this trial; the scale was designed to limit subjective scale and focus on functional aspects of the disease so that significant changes would be considered clinically meaningful.

The SCA3 genotype analysis also showed a reduction in the relative risk of falls in troriluzole-treated patients versus placebo (nominal p=0.04). Patient reported falls, as measured by adverse events reveal an approximately 50% reduction of fall risk in the troriluzole group (16% versus 32% AE incidence of falls in the troriluzole and placebo groups, respectively).

Across all genotypes, patients who were able to ambulate at baseline (i.e., f-SARA Gait Item score=1) showed a reduction in the relative risk of falls in troriluzole-treated patients versus placebo. Patient reported falls, as measured by adverse events reveal an approximately 59% reduction of fall risk in the troriluzole group (10% versus 23% AE incidence of falls in the troriluzole and placebo groups, respectively; nominal p=0.043). Overall, troriluzole demonstrated a favorable safety and tolerability profile, consistent with past clinical trial experience.

Throughout this application, various publications are referenced by author name and date, or by patent number or patent publication number. The disclosures of these publications are hereby incorporated in their entireties by reference into this application in order to more fully describe the state of the art as known to those skilled therein as of the date of the invention described and claimed herein. However, the citation of a reference herein should not be construed as an acknowledgement that such reference is prior art to the present invention.

Those skilled in the art will recognize, or be able to ascertain using no more than routine experimentation, numerous equivalents to the specific procedures described herein. Such equivalents are considered to be within the scope of this invention and are covered by the following claims. For example, pharmaceutically acceptable salts other than those specifically disclosed in the description and Examples herein can be employed. Furthermore, it is intended that specific items within lists of items, or subset groups of items within larger groups of items, can be combined with other specific items, subset groups of items or larger groups of items whether or not there is a specific disclosure herein identifying such a combination.

1. A method for treating spinocerebellar ataxia genotype type 3 in a patient in need thereof, comprising administering to the patient a dosage form comprising an effective amount of troriluzole hydrochloride monohydrate.

2. The method of claim 1, wherein the dosage form comprising 200 mg of troriluzole hydrochloride monohydrate is administered to the patient daily for forty eight weeks, and wherein the patient at week 48 showed a least squares [LS] mean change difference of −0.55 compared to placebo (nominal p-value=0.053, 95% CI: −1.12, 0.01).

3. (canceled)

4. The method of claim 1, wherein the dosage form is in the form of a capsule or tablet.

5. (canceled)

6. The method of claim 4, wherein the dosage form is in the form of an orally disintegrating tablet.

7. The method of claim 1, wherein the dosage form comprises 200 mg of troriluzole hydrochloride monohydrate, and wherein the dosage form is administered to the patient daily.

8. The method of claim 1, wherein the dosage form comprises 100 mg of troriluzole hydrochloride monohydrate, and wherein the dosage form is administered to the patient twice a day.

9. The method of claim 1, wherein the dosage form is administered daily for at least four weeks.

10. The method of claim 9, wherein the dosage form comprising 200 mg of troriluzole hydrochloride monohydrate is administered to the patient daily for four weeks, and wherein the patient at week 4 showed a least squares [LS] mean change difference of at least −0.46 compared to placebo.

11. (canceled)

12. The method of claim 1, wherein the dosage form is administered daily for at least eight weeks.

13. The method of claim 12, wherein the dosage form comprising 200 mg of troriluzole hydrochloride monohydrate is administered to the patient daily for eight weeks, and wherein the patient at week 8 showed a least squares [LS] mean change difference of at least −0.12 compared to placebo.

14. (canceled)

15. The method of claim 1, wherein the dosage form is administered daily for at least twelve weeks.

16. The method of claim 15, wherein the dosage form comprising 200 mg of troriluzole hydrochloride monohydrate is administered to the patient daily for twelve weeks, and wherein the patient at week 12 showed a least squares [LS] mean change difference of at least −0.46 compared to placebo.

17. (canceled)

18. The method of claim 1, wherein the dosage form is administered daily for at least twenty-four weeks.

19. The method of claim 18, wherein the dosage form comprising 200 mg of troriluzole hydrochloride monohydrate is administered to the patient daily for twenty-four weeks, and wherein the patient at week 24 showed a least squares [LS] mean change difference of at least −0.17 compared to placebo.

20. (canceled)

21. The method of claim 1, wherein the dosage form is administered daily for at least thirty-six weeks.

22. (canceled)

23. The method of claim 21, wherein the dosage form comprising 200 mg of troriluzole hydrochloride monohydrate is administered to the patient daily for thirty-six weeks, and wherein the patient at week 36 showed a least squares [LS] mean change difference of at least −0.55 compared to placebo.

24. The method of claim 1, wherein the dosage form is administered daily for at least forty-eight weeks.

25. The method of claim 24, wherein the dosage form is administered daily for an additional forty-eight weeks.

26. A dosage form comprising troriluzole hydrochloride monohydrate in an amount effective to treat spinocerebellar ataxia genotype type 3 in a patient in need thereof.

27. The dosage form of claim 26, comprising troriluzole hydrochloride monohydrate in an amount of 100 mg or greater.

28. (canceled)

29. (canceled)
